(19) United States
(12) Patent Application Publication
(10) Pub. No.: US 2020/0185070 A1
(43) Pub. Date: Jun. 11, 2020

(54) SELF-EXECUTING AGENTS FOR GATHERING HEALTH INFORMATION BETWEEN TRUSTED PARTIES

(71) Applicant: QuiO Technologies LLC, Chicago, IL (US)

(72) Inventor: Alexander Dahmani, New York, NY (US)

(21) Appl. No.: 16/637,123

(22) PCT Filed: Aug. 8, 2018
(86) PCT No.: PCT/US18/45708

§ 371 Date: Feb. 6, 2020

Related U.S. Application Data
(60) Provisional application No. 62/542,359, filed on Aug. 8, 2017.

(51) Int. Cl. G16H 10/60; G06F 21/62; G06F 21/60; G06Q 20/08

(52) U.S. Cl. CPC G16H 10/60; G06Q 20/08; G06F 21/602; G06F 21/6245

(57) ABSTRACT

Apparatuses, systems, methods, and computer readable media for processing performance-based healthcare payments are disclosed herein.

### PRIORITY CLAIM

This application is a 35 U.S.C. § 371 U.S. National Stage Application of International Application No. PCT/US2018/045708 filed Aug. 8, 2018, which claims priority to U.S. Provisional Patent Application Ser. No. 62/542,359, filed Aug. 8, 2017, the entire contents of which are incorporated herein by reference and relied upon.

### TECHNICAL FIELD

The present technology relates generally to processing and transmitting healthcare information and associated systems and methods. In particular, several embodiments are directed to automatically acquiring and processing information associated with a performance-based healthcare contract.

### BACKGROUND

Processing health information is done in many ways and for a variety of reasons. For example, health information is acquired and stored as a part of medical records. Such medical records can be used by healthcare professionals to provide higher quality treatment, because the healthcare professional will have information from the records about a patient's medical history. Medical records may also be shared between healthcare providers to improve care for a patient. Medical records also include confidential information about patients. Such information must be stored securely. Health information can also be acquired from patient monitoring devices, such as blood glucose meters, and insurance claims, such as pharmacy refills.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent. For ease of reference, throughout this disclosure identical reference numbers may be used to identify identical or at least generally similar or analogous components or features.

FIG. 1 is a block diagram illustrating computing devices and a server in accordance with an embodiment of the present technology.

FIG. 2 is a block diagram illustrating a network of computing devices capable of executing smart contracts in accordance with an embodiment of the present technology.

FIG. 3 is a block diagram illustrating software agents on computing devices in accordance with an embodiment of the present technology.

FIG. 4 is a flow diagram illustrating a method for processing performance based healthcare contracts in accordance with an embodiment of the present technology.

### DETAILED DESCRIPTION

The present technology is directed to apparatuses, systems, methods, and computer readable media for processing payments based on one or more predefined patient health-related data and/or metrics are described. In an embodiment, payments are exchanged between two parties when a shared patient's health-related metrics meet previously agreed upon conditions. The agreed upon conditions are incorporated into the logic of a computer-implemented smart contract, enabling payments to be automatically calculated and exchanged. In other words, the terms of the contract are built into software logic, such that when certain conditions are satisfied, the contract can be executed by the apparatus, system, method, or computer readable media without further input from a user. In this way, the contracts disclosed herein are described as self-executing. Once the agreed upon conditions are assented to by each party, the smart contract queries patient health-related data to determine when the conditions have been met. In addition, a network of multiple computing devices can be used to manage such a contract. Such a computer-implemented method and system improves inter-party trust through transparency, payment accuracy through consensus, and administrative efficiency through automation, reducing the time and resources required to execute a performance-based payment scheme.

In various embodiments, certain entities in health care seek to tie health benefits to payments and prices. Health benefits are defined as improvements in patient health-related metrics. For example, an in improvement in health-related metrics may be a cholesterol level or metrics that show a patient is using a medication as directed. Accordingly, certain entities may seek to, for example, reward patients or healthcare providers that show improvement in such health-related metrics. Implementation of such health benefits and rewards can be complex, expensive, and time-consuming. A goal of this technology is to reduce the time and resources needed to implement a performance-based payment system in healthcare, while improving the transparency and accuracy of payments.

In an example embodiment, a system may collect patient health data from external sources. The system packages said patient health data with instructions for calling one or more functions of a software contract. The instructions to call a function of a software contract occur when a condition set in one of the terms of the contract is met. Whether a condition in the contract is met depends on patient health data (e.g., cholesterol level, usage of meds). Such patient health data can be packaged together and addressed to the software contract.

If the condition is met, the software contract will recognize that the condition is met, and the software contract proceeds to self-execute based on the patient health data. Within the software contract, there is data that indicates one or more functions that determines, for example, an appropriate payment based on the input patient health data. In various embodiments, the action triggered may be something other than determining and initiating a payment. For example, the system may initiate some other type of reward or benefit in addition to or instead of a payment.

Such a software contract as described herein is sufficient to process a payment based on the input patient health data and instructions packaged by the computer program that make up the smart contract. To do so, the smart software contract code includes logic that embodies the payment terms agreed upon or assented to between at least two parties. While the terms of the contract must be assented to by both parties, the execution of the contract and subsequent output payment is dependent on the value of said input patient health data fulfilling conditions specified in the logic of the smart software contract code.

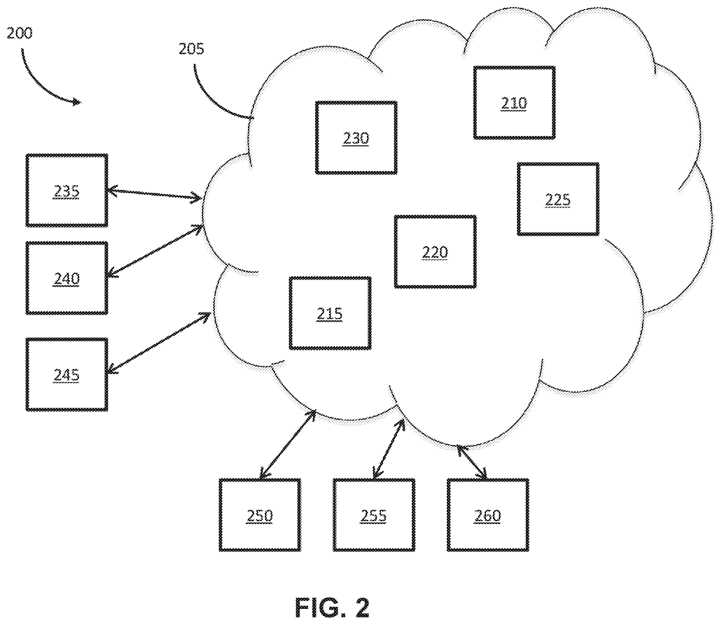

As disclosed herein (see FIG. 2 and associated description below), a network of computing devices can be advantageously used as disclosed herein. On a network of computing devices that manage the smart contracts, identical copies of the smart software contract are distributed and stored across a network of computers. The network of computers must achieve consensus regarding the software contract code in order for the contract to self-execute. Consensus rules can be set up differently in various embodiments. For example, full consensus of all devices may be used, or a threshold less than all devices agreeing can yield consensus. To reach agreement with each other for consensus, the network of devices must each receive the assent of the parties to the contract, and then must each receive the patient health data that indicates that the contract can self-execute. By requiring some consensus of multiple devices in the network, security is increased.

Therefore, each copy of the software contract on the various devices in the network receives the same input health data from the same source. The output payment resulting from self-execution of the software contract is also stored on a shared ledger across said network of computers. That is, each of the computing devices in the network store information indicating that a payment was made or ordered based on a particular contract and particular patient health data received. The patient health data and/or payment information may also be added to or written onto the contract on the various devices in the network using blockchain technology. In some embodiments, the smart contract does not initiate a payment transfer itself, but rather orders another entity (e.g., bank, healthcare provider accounting department) to make the payment. Such orders may be processed automatically, such that the self-executing contract causes a payment to occur without further intervention by a user after the initial smart contract is assented to by all parties to the contract.

Accordingly, the network of computers achieves consensus regarding the output payment from each copy of the software contract on each computer in order for the payment to be added to the shared ledger. The network can also support multiple distinct software contracts on the same network of computers. Different software contracts can be programmed to only accept input health data from specific software programs. For example, a contract specifying a reward for a certain average blood pressure may be configured to only accept data from a software program that works with blood pressure measuring devices. This adds another layer of security, so that only the specific conditions laid out in a contract can be met in order to have a contract automatically execute. In various embodiments, a single computer program can successfully package and address input patient health data to multiple distinct software contracts. For example, the blood pressure device can send data relating to multiple patients that each have a contract relating to their blood pressure. The system can receive the data and determine which patients' contract conditions were met, and which were not. Another security measure that can be used is encryption. A program sending patient health data can encrypt the data to protect it and ensure that only the target software contract can decrypt and use the data.

Patient health data can be collected from a variety of external sources. For example, patient health data may be collected from a patient monitoring device, mobile health application, clinical software system, claims software system, or patient registry. The patient health data collected includes at least one patient health-related metric. The health-related metric may be one or more of the following: medication adherence data, physiological monitoring data, diagnostic lab data, patient survey data or healthcare utilization data. This patient health data can be collected through periodic querying of the external sources that collect the data. Some patient health data may be processed before the querying at the external sources so that the data is in a particular form that can be understood by the software contract (and its agents/oracles). In some embodiments, the patient health data may be processed into a proper form at a device where the software contract (and its agents/oracles) are stored. In this way, the smart contract can seek out the information used to determine if the contract can be executed without further intervention or input from a user or the computing devices associated with the users that originally indicated assent to the contract. The software contract can then release payment to the appropriate party or order such a payment to take place as long as the conditions of the contract are met. Such an order may be sent to a different entity that processes such payments.

Such methods, systems, and computer readable media have several benefits and advantageous features. Using software to automate the labor-intensive and error-prone process of adjudicating payments, especially performance-based payments which are much more complex than regular claims or even rebate-based payments. The number of staff working on checking patient data and updating contract systems can be reduced. Distributing software across a network of computers also helps ensure accurate payment processing via consensus among many devices (avoiding errors and disputes). Storing transactions on an immutable or unchangeable ledger distributed across the network of computers also increases auditability and availability of records to all parties, payers, payees, etc. Such systems also allow involved parties to create and update performance-based contracts without impacting payment system or infrastructure, and parties can view the software program and ledger to ensure transparency. The systems and methods herein also increase privacy, as patient identities and other confidential information can be removed prior to health data transaction. In particular, when a query is sent for patient health data, only the relevant data value can be sent back without the need for transmitting personal information of the patient. That is, since the contract was already set up and agreed to previously, the device sending the query and the device answering the query only need to know the contract being referred to and the answer to the query. Personal information of the parties does not necessarily need to be transmitted. Additionally, each party can be configured to have a trusted identity, such that various transacting parties can be identified only by a number. In this way, an entity does not have to transmit identifiable information each time they assent to or create a new contract. Such entities can therefore be assigned a key that can be shared to identify themselves for entering into various smart contracts.

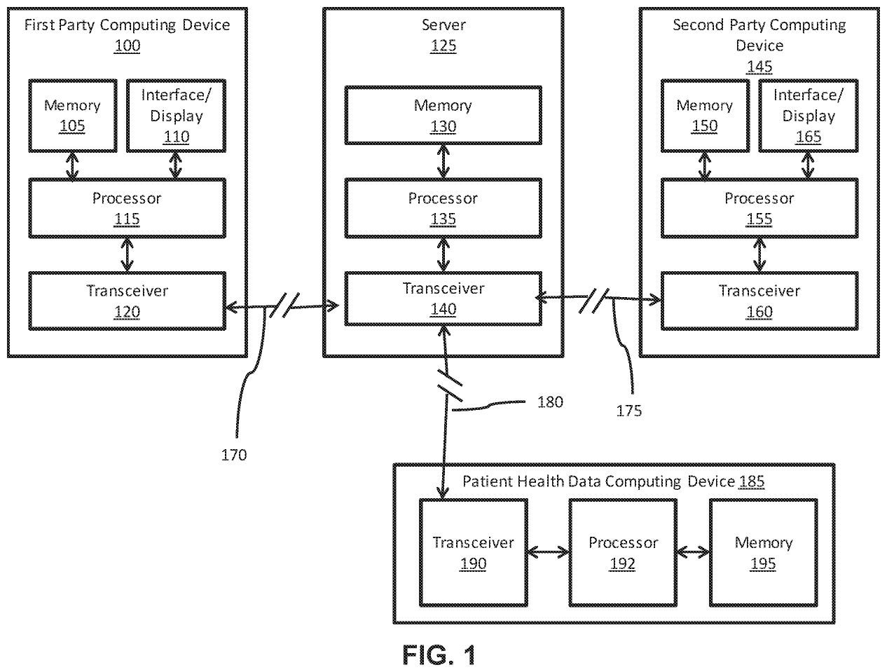

Specific details of several embodiments of the technology are described below with reference to FIGS. 1-4. Although many of the embodiments are described below with respect to devices, systems, methods, and computer readable media for processing performance-based healthcare payments, other applications and other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-4.

FIG. 1 is a block diagram illustrating computing devices and a server in accordance with an embodiment of the present technology. In various embodiments, fewer, additional and/or different components may be used in a system. FIG. 1 illustrates a first party computing device 100, a second party computing device 145, a server 125, and a patient health data computing device 185 that may be used in accordance with an illustrative embodiment. In alternative embodiments, fewer, additional, and/or different components may be included in the system. The computing device 100 includes a processor 115 that is coupled to a memory 105. The processor 115 can store and recall data and applications in the memory 105, including applications that process and utilize smart contract as disclosed herein. The processor 115 may also display objects, applications, data, etc. on the interface/display 110. The processor 115 may also receive inputs through the interface/display 110. The processor 115 is also coupled to a transceiver 120. With this configuration, the processor 115, and subsequently the first party computing device 100, can communicate with other devices, such as the server 125 through a connection 170. For example, the first party computing device 100 may send to the server 125 an assent to terms of a smart contract as disclosed herein.

The server 125 includes a processor 135 that is coupled to a memory 130. The processor 135 can store and recall data and applications in the memory 130. The processor 135 is also coupled to a transceiver 140. With this configuration, the processor 135, and subsequently the server 125, can communicate with other devices, such as the first party computing device 100 through a connection 170, the second party computing device 145 through a connection 175, and the patient health data computing device 185 through a connection 180.

Similar to the first party computing device 100, the second party computing device 145 includes a processor 155 that is coupled to a memory 150. The processor 155 can store and recall data and applications in the memory 150. The processor 155 is also coupled to a transceiver 160. The processor 155 may also display objects, applications, data, etc. on the interface/display 165. The processor 155 may also receive inputs through the interface/display 165. With this configuration, the processor 155, and subsequently the second party computing device 145, can communicate with other devices, such as the server 125 through the connection 175.

The patient health data computing device 185 includes a transceiver 190, processor 192, and memory 195 that can function similarly to similar components of the other devices. In this way, the patient health data can be identified in order to self-execute contracts at the server 125. Although not shown in FIG. 1, the patient health data computing device may have various types of inputs, such as a touch screen, medical sensor, or any other type of input through which patient health data can be input and/or measured.

The devices shown in the illustrative embodiment may be utilized in various ways. For example, any of the connections 170, 175, and 180 may be varied. Any of the connections 170, 175, and 180 may be a hard wired connection. A hard wired connection may involve connecting the devices through a USB (universal serial bus) port, serial port, parallel port, or other type of wired connection that can facilitate the transfer of data and information between a processor of a device and a second processor of a second device. In another embodiment, any of the connections 170, 175, and 180 may be a dock where one device may plug into another device. While plugged into a dock, the client-device may also have its batteries charged or otherwise be serviced. In other embodiments, any of the connections 170, 175, and 180 may be a wireless connection. These connections may take the form of any sort of wireless connection, including but not limited to Bluetooth connectivity, Wi-Fi connectivity, or another wireless protocol. Other possible modes of wireless communication may include near-field communications, such as passive radio-frequency identification (RFID) and active (RFID) technologies. RFID and similar near-field communications may allow the various devices to communicate in short range when they are placed proximate to one another. In an embodiment using near field communication, two devices may have to physically (or very nearly) come into contact, and one or both of the devices may sense various data such as acceleration, position, orientation, velocity, change in velocity, IP address, and other sensor data. The system can then use the various sensor data to confirm a transmission of data over the internet between the two devices. In yet another embodiment, the devices may connect through an internet (or other network) connection. That is, any of the connections 170, 175, and 180 may represent several different computing devices and network components that allow the various devices to communicate through the internet, either through a hard-wired or wireless connection. Any of the connections 170, 175, and 180 may also be a combination of several modes of connection.

To operate different embodiments of the system or programs disclosed herein, the various devices may communicate in different ways. For example, the computing device 100 and computing device 145 may download various software applications from the server 125 through the internet. Such software applications may allow the various devices in FIG. 1 to perform some or all of the processes and functions described herein. In another embodiment, the computing devices 100 and 145 may operate using internet browsers that can access websites that perform the functionality of any of the systems and methods disclosed herein. Additionally, the embodiments disclosed herein are not limited to being performed only on the disclosed devices in FIG. 1. It will be appreciated that many various combinations of computing devices may execute the methods and systems disclosed herein. Examples of such computing devices may include smart phones, personal computers, servers, laptop computers, tablets, blackberries, RFID enabled devices, or any combinations of such devices.

The configuration of the devices in FIG. 1 is merely one physical system on which the disclosed embodiments may be executed. Other configurations of the devices shown may exist to practice the disclosed embodiments. Further, configurations of additional or fewer devices than the ones shown in FIG. 1 may exist to practice the disclosed embodiments. Additionally, the devices shown in FIG. 1 may be combined to allow for fewer devices or separated where more than the four devices shown exist in a system.

FIG. 2 is a block diagram 200 illustrating a network 205 of computing devices capable of executing smart contracts in accordance with an embodiment of the present technology. In various embodiments, fewer, additional and/or different components may be used in a system. The network 205 includes devices 210, 215, 220, 225, and 230. These devices in the network 205 can serve as the multiple devices on which a smart contract is maintained and executed as disclosed herein. For example, the devices 210, 215, 220, 225, and 230 may each be similar to the server 125 in FIG. 1. As disclosed herein, consensus of the devices can be used to increase security, transparency, and accuracy of smart contracts. There can be more or less devices in the network 205 than what is shown in FIG. 1.

Devices 235, 240, and 245 are party devices that can send signals to the devices 210, 215, 220, 225, and 230 in the network 205 to assent to contracts. There can be any number of party devices associated with any number of parties. Some devices may be associated with more than one party. These party devices may be similar to the first party computing device 100 and the second party computing device 145 shown in FIG. 1. In some contracts, certain parties may not be involved in that particular contract. Devices 250, 255, and 260 show patient health data computing devices. Such devices may be a patient monitoring device, mobile health application, clinical software system, claims software system, a patient registry, or any other type of device that can store and/or collect patient health data. These devices may be similar to the patient health device 185 shown in FIG. 1. These devices can be queried by the devices maintaining smart contracts in the network 205 for patient health data that may satisfy conditions of the terms various smart contracts. There may be more or less patient health data devices than the three shown in FIG. 2. In addition, some of the devices may serve multiple functions. For example, two parties may consent to a contract using the same device. In another example, a party may assent to a contract on a first device, and patient health data from that same first device may be relied upon to execute a contract on a second device.

The network of computers running and/or storing a smart contract can be very large, such as a Bitcoin or Ethereum network, or as small as three nodes run by a contract's participants. Tamper resistance in a three node system is achieved when only one of the contract participants cannot stop the smart contract from self-verifying and/or self-executing. A value is provided by larger networks because it is considered nearly impossible that a single contract participant could influence a network of, for example, 6000+ nodes exclusively in its favor. Accordingly efficiencies are created by smart contracts in industries such as health care where accurate monitoring and execution of high-value contracts is critical. Insurance, derivatives, and trade finance are other industries that could benefit due to the extremely low cost of servicing a fully automated smart contract as disclosed herein. In particular, instead of having multiple people in separate departments check a website or call a data source for proof of performance (e.g., price, weather, location, etc), the contract itself is can automatically check that same proof of performance as disclosed herein.

Smart contracts can also be referenced by multiple private systems at the various companies that are contract participants, making the smart contract a single point of truth that is tamper resistant and can be relied upon to trigger payment, accounting and compliance events for internal systems.

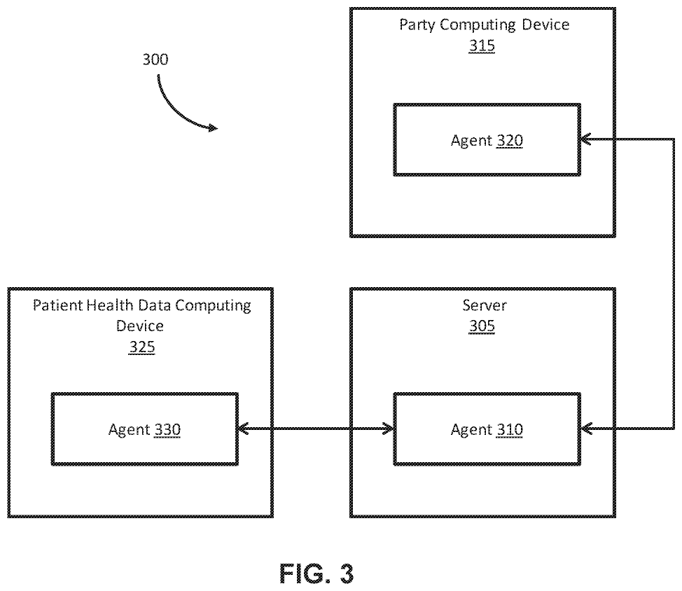

FIG. 3 is a block diagram 300 illustrating software agents on computing devices in accordance with an embodiment of the present technology. In various embodiments, fewer, additional and/or different components may be used in a system. In various embodiments, the systems, methods, and computer readable media disclosed herein can function utilizing agents (also referred to herein as oracles).

The smart contract network can be configured to have an inherent input/output limitation due to security constraints that limit the ability of the system to access anything outside its known or trusted network. Because critical data about contractual performance (e.g., cholesterol level, usage of meds, etc.) and the forms of payment preferred by recipients are currently outside smart contract networks, a secure point of connection between smart contracts and these external resources can be utilized.

Oracles or software agents can be used to provide information from outside a system which that system itself cannot acquire. When applied to the smart contract networks disclosed herein, oracles act as programmable agents that provide a smart contract with the data it requires (inbound oracles) and act on its behalf by releasing payment or informing your internal systems what a smart contract has concluded (outbound oracles).

Instead of a smart contract initiating the retrieval of external data, one or more trusted parties (“oracles”) creates a transaction which embeds that data in the chain. Every node will have an identical copy of this data, so it can be safely used in a smart contract computation. In other words, an oracle pushes the data onto the blockchain rather than a smart contract pulling it in. Inbound oracles provide smart contracts with data from external data feeds so they can make a determination about events outside the smart contract network in which they are required to run.

Outbound oracles allow smart contracts to send commands to your internal systems and traditional payment methods that release payment to a recipient in their preferred local currency. Private smart contract data storage happens as a natural consequence of using oracles to interact with external resources. If a data feed provides a large result with hundreds of values to create context and fully prove contractual performance, then the oracle provably records and retains all of this highly detailed data, but can be preset to pass in only the most critical value that proves performance into the smart contract (e.g., cholesterol level).

Accordingly, FIG. 3 shows a representation of a party computing device 315 running an agent (or oracle) 320. The agent 320 can communication with an agent 310 running on a server 305. The agent 310 can also communicate with an agent 330 running on a patient health data computing device 325. By using a similar agent or oracle on each device, the system ensures that each device is associated with a trusted party and can limit the amount of data that is transmitted between devices for every smart contract that is initiated and/or executed.

By retaining the larger data set that proves contractual performance with context (e.g., timestamp, signatures, device ID, data provider name, etc.) and passing only the most critical limited data into the smart contract (e.g., a single number for price, true/false, etc.), we reduce the amount of data fed into the smart contract. Since the smart contract receives only the critical data it needs to make a determination, it is able to retain a high level of privacy; there is no way to tell what the smart contract code relates to outside of the network, so that even if someone does view the smart contract, they cannot acquire any valuable information from it. By presetting the oracle to pass in only the most critical information, we have nearly eliminated all information about the smart contracts context/purpose, providing it with a high degree of privacy while running in a large network. Smart contracts run on node networks beyond the control of a contract's participants, assuring that the contract will be executed as it was written once performance occurs. Contracts are also self-verifying in that they can be evaluated and are provable based on the recording that contractual performance has occurred and the patient health data that is recorded as a part of that record. Recording this performance in real time as it is reflected in various pre-defined data feeds (e.g., cholesterol level, usage of meds, events, etc.) also increases accuracy and transparency to participants or parties in the system. For example, only after a condition is provably met (and consensus achieved), will a contract self-execute and initiate an action between parties.

Smart contracts are uniquely tamper resistant because they are run and/or stored on a network of computers that is beyond the influence of the contract's participants. Since none of the contract's participants can influence execution of the smart contract beyond actual performance of their obligations, all of the participants can trust that this type of contract will be executed as it was written. However, some systems may be equipped with methods for parties to a contract to mutually agree to terminate the contract and/or assent to new/different terms.

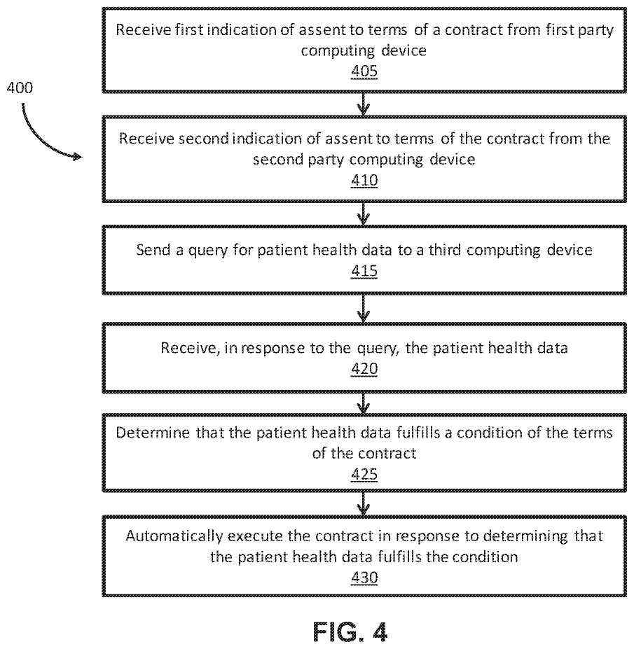

FIG. 4 is a flow diagram illustrating a method 400 for processing performance based healthcare contracts in accordance with an embodiment of the present technology. In various embodiments, fewer, additional, and/or different operations may be performed. Also, the use of a flow diagram is not meant to be limiting with respect to the order of operations performed unless otherwise noted. In an operation 405, the system receives a first indication of assent to terms of a contract from a first computing device associated with a first party to the contract. In an operation 410, the system receives a second indication of assent to the terms of the contract from a second computing device associated with a second party to the contract. In this way, the system can establish a contract to be executed by receiving data indicative that each party has consented to the contract. In some embodiments, the assent of the parties may be packaged together with the terms of the contract when sent to a server or network of nodes.

In an operation 415, the system sends a query for patient health data to a third computing device. Here, the system is attempting to check to see if patient health data is sufficient to self-execute the contract. In an operation 420, the system receives, in response to the query, the patient health data. In some embodiments, the patient health data may include health data on multiple patients and/or related to multiple contracts. Such data may be packaged by a single source device to reduce traffic and total number of communications utilized to administrate a large number of smart contracts by the network. These queries may also be sent periodically, so that the system can stay updated with new patient health data as that data is acquired and the contracts can continue to self-execute over time. In an operation 425, the system determines that the patient health data fulfills a condition of the terms of the contract. In an alternative embodiment, the system may determine that the patient health data does not fulfill the terms of the contract. In such an embodiment, the system may query for the information again later, or the system may terminate the contract if the terms of the contract dictate such an action.

In an operation 430, where the patient health data successfully fulfilled the condition of the contract, they system automatically executes the contract in response to that determination. In various embodiments, the execution involves initiating or ordering a payment between parties to the smart contract. For example, this automatic execution of the contract may include sending a payment initiation message that instructs a payment to be made in accordance with the terms of the contract. Further, as discussed herein, the contract can be self-executing such that a payment initiation message is sent without further interaction from the first computing device and the second computing device.

In some embodiments, the computing device from which the patient health data is from is different from computing devices that are associated with the parties of the contract. For example, the patient health data may have come from a heart rate monitor, while the consent of the parties may have come from a laptop computer at a doctor's office. The system can also update the contract to include a record that the contract has been executed, wherein the record is immutable, similar to blockchain technology. This creates a record of the contract terms and how/when/etc. it was executed, and the record can be accessed by the parties through the network of devices that administrated the contract.

As described herein, the system advantageously checks for patient health data without user interaction so that contracts can self-execute. Accordingly, with respect to the flow diagram 400, the system can perform each of sending the query (operation 415), receiving the patient health data (operation 420), and determining that the patient health data fulfills the condition (operation 425) without interaction with parties to the contract or computing devices associated with the parties.

In a specific embodiment, a stakeholder is a pharmaceutical organization. Payments are meant to be distributed from a health insurer to the stakeholder (pharmaceutical organization) that represent compensation for dispensed medications that lead to improvements to one or more patient health-related metrics. In other words, if dispensed medications work to improve a patient's condition, a contract can dictate that the insurer owes additional money for successful use of the drug. On the other hand, payments can be distributed from the stakeholder to the health insurer as rebates for purchased medications that did not lead to improvements to one or more health-related metrics of a patient. Therefore, the smart contracts as disclosed herein can be used to administrate such a contract, because the system can continuously monitor various systems that have data relating to the patient's condition to determine if the drug improved or did not improve the patient's condition.

In another embodiment, a stakeholder is a healthcare provider. In an example, smart contract payments are to be distributed from a health insurer to the stakeholder (healthcare provider) that represent compensation for activities that lead to improvements to one or more health-related metrics of a patient. For example, clinicians may be reimbursed for providing appropriate or effective care, and health coaches may be paid for providing remote support In the health coach example, payment may be tied to productivity while using tracking software and/or improvements in a supported patient's health-related metrics. On the other hand, payments may be distributed from the stakeholder to the health insurer as rebates for reimbursed activities that did not lead to improvements to one or more health-related metrics. Such a smart contract can also be administered efficiently utilizing the systems and methods disclosed herein.

In another embodiment, the stakeholder is the patient. In such a smart contract, payments would be distributed from the health insurer to the stakeholder as compensation for patient activities that lead to improvements to one or more health-related metrics. For example, a patient may be prescribed a weight-loss drug (e.g., Contrive), and the smart contract can set up payments or micro-incentives for adherence to drug use guidelines. Other micro-incentives for weight loss (as measured by home measurements and/or clinical visits) or exercise (as measured by fitness tracker data) may be offered through another smart contract.

In such smart contracts, payment terms are agreed upon, explicitly or implicitly, between the transacting parties. Patient inclusion criteria may be used to determine eligibility to participate in a smart contract, such as a verified diagnostic code or health-related metric, plus a verified patient health endpoint that would result in performance under the contract, such as a health-related metric or threshold. Appropriate patient inclusion criteria will help avoid unnecessary procedures that end up performing well (e.g., healthy people getting heart surgery inappropriately and surviving) or including patients on contracts they could never perform under (e.g., a person who is not on any medications being on a medication adherence plan). A patient inclusion criteria may include one or more of the following: a medical diagnosis, prescribed medication, medical procedure, or demographic feature.

Appropriate patient health endpoints can help identify necessary treatments or procedures that are handled improperly and don't end up performing well. A patient health endpoint may be associated with the safety and/or efficacy of a medical treatment, procedure, and/or patient behavior. Patient health endpoints include a quantifiable health-related metric that can act as input into a smart contract code. Health related metrics used in a smart contract could be adherence rate, blood glucose, fitness activity, weight, LDL cholesterol, or any other health metric. The patient health endpoint includes one or more clinically relevant parameters associated with the health-related metric. The parameter(s) could be an absolute value, a single-bound threshold, a double-bound range, or a magnitude of change over time (e.g., 80% adherence threshold, 7% HbA1c threshold, range of acceptable blood glucose, blood pressure, or LDL levels). When a parameter applies to one measurement of a given health-related metric at a designated time a party may be deemed to have performed under the terms of the smart contract. In another embodiment, a party may be considered to have performed when a quantified threshold applies to multiple measurements of a given health-related metric over a period of time. In some embodiments, a quantified threshold may apply to multiple health-related metrics.

Various sources may be used for patient healthcare data as described herein. The source of patient data should be sufficient to provide patient inclusion criteria and patient health endpoints. An internet-connected computer system with digital forms of patient health-related metrics, which when properly structured and injected into the network of distributed smart contract nodes, represents an input into the logic of the smart contract code that can be sufficient.

Actions as a result of an executed contract can also vary. For example, the action may be a payment, and the payment amount can be a set amount, follow a pricing schedule, or be calculated proportional to the health-related metric value that is used to determine performance under the smart contract (or even a metric that is not used to determine performance). Payments may also use different currency. For example, a transaction encompasses exchange of digital asset (e.g. cryptocurrency). In another example, a digital asset may be held until a specified performance threshold is achieved, and parties can utilize a digital wallet for receiving, holding, and sending the digital asset. Financial payments may also be made periodically by digital asset amounts to a government-backed currency. A transaction may also encompass a digital exchange of government-backed currency held in escrow between two parties.

Each contract may have terms including payment terms between two transacting parties, patient inclusion criteria, quantified health data thresholds (endpoints) associated with the efficacy of a medical treatment, procedure, and/or patient behavior, and any other data relating to the contract. These terms can be viewable between transacting parties on their respective devices both before and after assenting to the contract. These terms can be viewed within the distributed software applications, in an encrypted fashion, in which only the transacting parties see the data and other nodes in the network are unable to view the details of the transaction and smart contract code. The type of software contract code could be, for example, Turing complete (e.g. Ethereum).

The system can also be connected to a payment network so that contracts can be effectively executed. Such payments may utilize a permissioned blockchain for security (a blockchain that requires users to be added by an administrator, and uses mining or a voting system to verify transactions, which are not necessarily incentivized with tokens). A peer-to-peer network with logic for executing smart contract code (e.g. Ethereum), distributed ledger for storing transactions (e.g. blockchain), mechanisms for achieving consensus between the nodes (e.g. proof of stake), and means for exchanging digital assets (e.g. cryptocurrency) can all be utilized to effectuate payments according to the smart contracts disclosed herein. The immutable transaction ledger (at least immutable to the parties of the contract) can also be made viewable between the parties involved in a transaction/contract.

Different mechanisms to build consensus on a distributed system such as that in shown in FIG. 2 can also be used. For example, simple majority (51% consensus), proof of work, compute-heavy, proof of stake, or absolute (100% consensus) standards may be used to determine consensus for a contract to proceed with self-executing. Custom standards for consensus may also be implemented, and may even be a term of the contract being agreed to.

The agents or oracles (types of middleware) used as described herein may also vary. Such middleware communicates with external databases containing patient records that serve as health data sources and identifies relevant patient records to pull health data from based on patient inclusion criteria. The agents or oracles can also pull in relevant health data in real-time or on a periodic basis, and can transforms the data into a structure appropriate for use by the smart contracts to properly determine whether performance of a contract has occurred. The middleware also ensures that the relevant health data based on payment terms, including patient inclusion criteria and health data thresholds, is acquired.

The inbound agent/oracle ensures the health data is structured and fed properly into the network of distributed smart contracts. This ensures input health data is standard and consistent while being consumed by the disseminated smart contract code, and that consensus can be properly built. If a smart contract is responsible for pulling in data from an external source, the data at the source may change over time between nodes running the contract, and the consensus mechanism could fail (chain will fork). However, the inbound agent/oracle can target a specific data package to an appropriate smart contract code, and in certain embodiments, a specific function within the smart contract code. Therefore, the inbound agent/oracle can be responsible for feeding data to a specific smart contract only for patients fitting the inclusion criteria specified in the payment terms (for optimal compute efficiency). In this way, forking can be avoided by reducing amount of data that is input to or sent to specific contracts to only data that is pertinent to those contracts. In some embodiments, the inbound agent/oracle simply feeds data to a specific smart contract based on the patient health endpoint, and the rules incorporated into the smart contract will prevent a successful transaction from occurring for patient data that does not fit the inclusion criteria (optimal simplicity). In some embodiments, both the inbound agent/oracle and the smart contract are able to determine whether the patient health data fits the patient inclusion criteria (optimal reliability). In some embodiments, the inbound agent/oracle also serves other applications such as web applications with dashboards for viewing health data.

In some embodiments, performance-based payments may be paired with traditional fee-for-service payments (e.g., fixed payments across all patients receiving the medical treatment). Performance-based payments may take time for an outcome to be achieved, requiring product (e.g., pharmaceutical company) or service (e.g., clinicians) providers to be paid a portion upfront. The technology embodied herein can determine, process, and exchange payments between the product/service provider and the health insurer. Bonuses for health improvement and rebates for outcomes below a defined threshold would be exchanged, helping to better link the overall payment (benchmark+performance) to the value delivered long-term, without adding significant administrative work for processing such bonuses. For example, such bonuses may be processed after normal data entry of a follow up appointment with a patient.

The system may also be used to incentivize patients or compensate remote caregivers for time spent on the platform or for performing tasks, in addition to health improvements. In other words, someone can get paid for amount of time spent in a connected software application, with bonuses paid later for health improvements. This can incentivize use of tools available and increase data entered by patients, even if it is not favorable (e.g., related to health improvements).

Alternative financial payments are also contemplated herein. For example a token payments and balances system mirrored by a traditional finance system may be used. For example, each party can receive a starting balance of tokens for free (e.g., 100). The tokens have no inherent value but are pegged to government backed currency. The tokens can be exchanged between parties and held in accounts on the network. The balance of each account is measured at periodic time (e.g. monthly) and translated into traditional billing/payment system between two parties. In this way, those with good health outcomes will amass more tokens and be rewarded accordingly. A token balance may reset after the period ends (e.g., back to 100). This system may be beneficial as a starting strategy when there is no liquidity (e.g., just a few parties involved in the system).

In another alternative, tokens may be purchased by involved parties using traditional currency (e.g. USD). These tokens can be held in a smart contract. An insurer and supplier (e.g., clinician or pharma) may put a max possible rebate/bonus owed into the smart contract and/or additional patient reward tokens in. Payments can then be exchanged using tokens when the smart contract is executed. A party that loses their tokens from smart contracts then purchase more tokens to participate in a new smart contract. A party that gained tokens from the smart contract has surplus and can sell tokens in exchange for traditional currency (i.e., cashing out). A patient therefore cashes out their rewards when insurer/suppliers buy them for future smart contracts. This is a good system for many users and true automation where there is liquidity (stakeholders will need to be able to sell tokens and cash out in order to run their business).

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A system comprising:
a memory; and
a processor coupled to the memory, wherein the processor is configured to:
receive a first indication of assent to terms of a contract from a first computing device associated with a first party to the contract;
receive a second indication of assent to the terms of the contract from a second computing device associated with a second party to the contract;
receive patient health data from a third computing device;
determine that the patient health data fulfills a condition of the terms of the contract; and
automatically execute the contract in response to determining that the patient health data fulfills the condition.

2. The system of claim 1, wherein, in order to receive the patient health data, the processor is further configured to:
send a query for the patient health data to the third computing device; and
receive, in response to the query, the patient health data.

3. The system of claim 1, wherein the processor is further configured to receive the patient health data as a result of the patient health data being pushed to the system.

4. The system of claim 1, wherein the first computing device is different from the second and third computing devices.

5. (canceled)

6. The system of claim 1, wherein the automatic execution of the contract further comprises sending a payment initiation message that instructs a payment to be made in accordance with the terms of the contract.

7. The system of claim 6, wherein the contract is self-executing such that the payment initiation message is sent without further interaction from the first computing device and the second computing device.

8. The system of claim 1, further comprising a network of computing devices, and wherein the network of devices must achieve at least partial consensus that the patient health data fulfills a condition of the terms of the contract before the contract is executed.

9. The system of claim 8, wherein each of the network devices that achieves the at least partial consensus receives the patient health data.

10. The system of claim 1, wherein the processor is further configured to update the contract to include a record that the contract has been executed, wherein the record is immutable.

11. The system of claim 1, wherein the patient health data further includes a plurality of patient health data packaged together and relating to a plurality of contracts.

12. The system of claim 11, wherein the plurality of patient health data is received from a single computing device source.

13. A non-transitory computer readable medium having instructions stored thereon that, upon execution by a computing device, cause the computing device to perform operations comprising:
receiving a first indication of assent to terms of a contract from a first computing device associated with a first party to the contract;
receiving a second indication of assent to the terms of the contract from a second computing device associated with a second party to the contract;
receiving patient health data from a third computing device;
determining that the patient health data fulfills a condition of the terms of the contract; and
automatically executing the contract in response to determining that the patient health data fulfills the condition.

14. The non-transitory computer readable medium of claim 13, wherein the patient health data is encrypted, and the contract includes instructions for decrypting the patient health data.

15. The non-transitory computer readable medium of claim 13, wherein the third computing device from which the patient health data is received comprises at least one of a patient monitoring device, a mobile health application, a clinical software system, a claims software system, and a patient registry.

16. (canceled)

17. The non-transitory computer readable medium of claim 13, wherein the patient health data comprises at least one of medication adherence data, physiological monitoring data, diagnostic lab data, patient survey data, and healthcare utilization data.

18. A method comprising:
receiving, by a processor of a computing device, a first indication of assent to terms of a contract from a first computing device associated with a first party to the contract;
receiving, by the processor, a second indication of assent to the terms of the contract from a second computing device associated with a second party to the contract;
receiving, by the processor, patient health data from a third computing device;
determining, by the processor, that the patient health data fulfills a condition of the terms of the contract; and
automatically executing, by the processor, the contract in response to determining that the patient health data fulfills the condition.

19. The system of claim 18, wherein, in order to receive the patient health data, the processor is further configured to:
sending, by the processor, a query for the patient health data to the third computing device; and
receiving, by the processor and in response to the query, the patient health data.

20. (canceled)

21. The method of claim 19, wherein each of sending the query, receiving the patient health data, and determining that the patient health data fulfills the condition occurs without interaction with the first computing device and the second computing device.

22. The method of claim 18, wherein the third computing device and the first computing device are the same, but the third computing device is different from the second computing device.

23. The method of claim 18, wherein the automatically executing the contract further comprises writing the patient health data into the contract itself.
